(19) United States
(12) Patent Application Publication
(10) Pub. No.: US 2019/0014842 A1
(43) Pub. Date: Jan. 17, 2019

(54) Garment With Sound Absorbing Layer

(71) Applicants: Tara A. Israel, East Hampton, NY (US); Bonnie S. Schnitta, East Hampton, NY (US)

(72) Inventors: Tara A. Israel, East Hampton, NY (US); Bonnie S. Schnitta, East Hampton, NY (US)

(21) Appl. No.: 16/124,806

(22) Filed: Sep. 7, 2018

Related U.S. Application Data
(63) Continuation-in-part of application No. 14/996,213, filed on Jan. 14, 2016.

(51) Int. Cl. A41D 31/00; A41D 1/04; A41D 23/00; A41D 3/08; A41D 15/00; A42B 1/00; B32B 1/00; B32B 5/02; A61F 11/14

(52) U.S. Cl. CPC A41D 31/0016; A41D 1/04; A41D 23/00; A41D 3/08; A41D 15/00; A42B 1/006; B32B 2437/04; B32B 5/024; A61F 11/14; A41D 2300/322; A41D 2300/32; A41D 2200/20; B32B 2307/102; B32B 1/00

(57) ABSTRACT

A garment engaged in a sound-reducing state includes a first garment portion formed with a sound-absorbing material with transmission loss properties and configured to surround a wearer's head and ears to form a substantially enclosed reduced-sound zone, when the first garment portion is engaged in a sound-reducing state. The garment also includes a second garment portion operable with the first garment portion, for example, where the first garment portion is a hood and the second garment portion is the body of a hooded sweatshirt. In the sound-reducing state, the sound-absorbing barrier material reduces an amount of sound passing through the sound-absorbing material and over the material on the inside of the reduced-sound zone into the substantially enclosed reduced-sound zone.

### CROSS-REFERENCE TO RELATED APPLICATIONS

This application is a continuation-in-part (CIP) application based on U.S. patent application Ser. No. 14/996,213, filed on Jan. 14, 2016; U.S. patent application Ser. No. 14/996,213 derives the benefit of the filing date of U.S. Provisional Patent Application No. 62/103,356, filed Jan. 14, 2015; The contents of U.S. patent application Ser. No. 14/996,213 and U.S. Provisional Application No. 62/103,356 are incorporated herein by reference.

### BACKGROUND OF THE INVENTION

The present invention is directed to garments formed with a lining of material that operates to absorb and attenuate sound that might otherwise pass through the lining of material, as well as absorb the sound as it passes over the inner section of the hood, scarf, etc.

Many garments are currently available with an added layer of material for a specific function, such as heat insulation or light reflection, when a basic garment without added features does not achieve desired technical or aesthetic goals. U.S. Pat. No. 8,625,812 (the '812 patent) discloses headwear that acoustically compensates or dampens detected acoustic signals in order to reduce the noise/sound that passes into a garment, such as a hooded sweatshirt. The acoustic management system of the '812 patent, however, is based on active noise cancellation and if the active noise cancellation is removed or underpowered, the hood would not and could not work as intended by the present invention.

U.S. Pat. No. 8,590,539 discloses a headgear-ear wear assembly but does not refer to reduction of ambient or other sound proximate a wearer/user, nor under its present design would it be capable of doing so.

US Published Application Serial. No. 2015/0041243 discloses a hearing protection system, which may be attached to a cap and requires effort to put into a position that would protect the ears from noise. It is also not easily disguised as a typical wearable garment so would not be desirable for daily use in public environments. Most important is that the known hearing protection system does not allow for one to be aware of ongoing activity while also reducing noise and therefore dangerous to wear as a pedestrian or while operating a vehicle. The hearing protection system is limited in utility because it is cumbersome and offers no reduced sound zone about the user's ears.

U.S. Pat. No. 9,190,045 discloses a noise absorbent fabric for vehicles. Such fabric is generally non-woven and not used for garments. Even if sewed into a garment, it would not function to provide a reduced sound zone without the complete design of this present invention. This patent and many more of this category address a pressure wave by some form of an active or electric processing system, whereas the present invention is a clever passive system of noise reduction. U.S. Pat. No. 9,186,092 discloses a system garment and method that incorporate sensors used to measure or monitor pressure or forces in feet, the stump of limbs of an amputee, etc.

U.S. Pat. No. 3,728,741 discloses a noise protective headgear 10 that is wrapped around a user's head. The headgear comprises a flat, pliant, elongate panel 11 that longitudinally extends to drawstrings that compel pliant sections to conform to the outside of the user's ear (FIG. 4). FIG. 4a highlights the material utilized. That is, layers of rayon or flexible polyurethane foam 17 are formed about a fibrous glass blanket layer 19. Tin-loaded vinyl septums 17, 20, are positioned between the opposing sides of the fibrous glass blanket 19 and the respective sheathing layers 17. The patent is designed to allow the communication of speech while attenuating higher frequency noise (col. 1, line 61-64).

### SUMMARY OF THE INVENTION

The present invention overcomes the shortcomings of known arts, such as those mentioned above to provide a simple but comfortable everyday garment to passively reduce or enhance sound impinging on the wearer's ears, including speech frequencies.

To that end, the present invention provides a garment that is arranged with a garment portion configured to form a reduced-sound inner volume around a wearer's head, in particular, including the wearer's ears, when the inner volume is substantially closed, for example, such as by tying drawstrings tight to substantially close the inner volume, or pulled forward to act as a muffler. The garment wearer/user organically experiences a sound-reduced state. Depending on the configuration of the material from which the garment is formed (e.g., layered), or a design of a specific garment, the invention offers the wearer with varying levels of sound reduction in addition to aesthetic or additional functions. The garment includes a first garment portion, such as a hood portion of a sweatshirt and a second portion, such as the trunk portion of the sweatshirt, which second garment portion is operable with the first garment portion.

In an embodiment, the invention provides a garment engaged in a sound-reducing state. The garment includes a first garment portion formed from a material that includes a sound-absorbing material. The first garment portion is configured to surround part of a wearer's head and form a substantially enclosed reduced-sound zone about the user's ears when the first garment portion is engaged in a sound-reducing state. A second garment portion is operable with the first garment portion, for example, the first garment portion and the second garment portion detachably connect, such as in a hooded sweatshirt, where the hood is detachable. A zipper, buttons and Velcro embody devices for the detachable connection. In the sound-reducing state, the sound-absorbing material reduces an amount of sound passing through the sound-absorbing material into the substantially enclosed reduced-sound zone.

The material forming the first garment portion preferably comprises a base layer upon which the sound absorbing material is provided. Alternatively, the material forming the first garment portion comprises a base layer treated with the sound absorbing material or, the material forming the first garment portion comprises a base layer and wherein the sound absorbing material is impregnated into or otherwise integrated with the base layer. For that matter, the base layer may be formed as a natural or synthetic material.

In one form, the garment is a hooded sweatshirt, where the first garment portion is a detachable hood and the detachable hood is deployed to form the substantially enclosed reduced-sound zone. The reduced-sound zone minimizes an amount of sound entering the reduced-sound zone from outside the garment and reduces an amount of sound exiting the zone from inside the garment. In one form, the first garment portion and the second garment portion comprise the same material.

In another embodiment, the garment includes small cups in or proximate the reduced-sound zone, for receiving and directing sound towards the wearer's ears. The small cups are arranged to be tight to the wearer's ear at the back of the small cups. The small cups are open to maintaining a space between a face of the wearer the garment in a direction that the wearer is facing, for receiving and amplifying sound directed to a wearer. In an alternative embodiment, the small cups are arranged in or proximate the reduced-sound zone, in a way that inhibits sound going towards the wearer's ears. Preferably, the small cups are arranged to be tight to the wearer's ear all around the small cups. The small cups, or passive amplifiers, are secured to the hood by Velcro, an internal band, or any other method that the user finds as convenient.

In another form, the first garment portion is a scarf that is engaged to form the substantially enclosed reduce-sound zone. In a variation, the scarf is part of another system, such as a hat, i.e., where the scarf comprises the first garment portion, which defines the inner, sound-reduced volume and the hat comprises the second garment portion, the hat and scarf embodying the inventive garment. In another embodiment, the garment is the hooded sweatshirt where the first garment portion is the hood that is substantially forward of the face creating a passive muffler. To that end, the hood can be closed, creating the substantially forward of the face creating the passive muffler.

The sound absorbing lining, or sheet material formed with the sound absorbing layer may be any type of material that is flexible and can be made into a wearable garment, including weaves and knits, as well as organic or inorganic polymer sheets.

The sound absorbing material that faces the ear may be any type of material that absorbs or inhibits sound energy when sound waves pass by or through the material, as opposed to reflecting or channeling the sound energy toward the ear. For example, LUMITEX® sound absorbing material, manufactured by or for, distributed and/or sold by SoundSense LLC, Wainscott, N.Y. LUMITEX® sound absorbing material is a lightweight, breathable, non-woven fabric that provides excellent acoustic absorption while requiring a fraction of the space required for traditional absorptive panels. The LUMITEX® sound absorbing material may also be used as a sound barrier, i.e., may be used as a sound absorbing material (e.g., a sound absorbing material layer) and/or as a sound barrier layer according to the invention. For example, multiple layers of sound absorbing material such as LUNTEX® may be used to form a laminate that operates as an excellent sound barrier/absorber.

Whereas a cotton or other similar thermoregulated material absorbs sound, it does not stop sound. That is, an absorber, such as fiberglass or cotton typically absorbs about 2-3 dB (when at the thickness of a hoodie), which is considered not a perceivable level of reduction. Cotton and other thermoregulated materials have NRC value, but not STC value. Even at the thickness of a wall cavity an absorber only absorbs about 5-7 dB.

A barrier on the other hand is governed by its ability to attenuate sound and has an STC rating, but typically not NRC. A barrier, such as a mass loaded vinyl, MLV, stops (depending on the density per square feet of the material) typically stops anywhere from 23 dB to 35 dB. The common MLV has a weight of one pound per square foot and an STC of 26. The problem is that to wear MLV on a head or jacket would be uncomfortable, due to the weight. Lumitex absorbs and stops sound and is a light fabric. A three-layer system, for example, having a base layer, a barrier layer provided thereon, where a sound absorbing layer is provided on the barrier layer is an inventive embodiment. For a garment, however, such a construction typically is heavier than two-layer solutions, so are preferred for limited applications.

The sound absorbing material is adhered to or otherwise arranged to be integral with at least a part of the material forming a garment, for example, a base layer, or alternatively, a layer of sound absorbing material adhered to or otherwise integral with a sound barrier layer. Then at least a part is arranged or configured to be closed to form the sound-reduced inner volume. In one form, the inventive sound attenuating cloth or material is formed from a base material (layer), such as a weave, where the sound-absorbing material is arranged on the base sheet, in a portion of the garment that will be proximate the ear and the area between the ear and the entrance area(s) of the acoustic wave. The sound absorbing material can be attached by any standard means of garment assembly. The sound-absorbing material may comprise a single layer knit or woven cloth formed using natural or polymeric (synthetic) materials or any other natural or synthetic materials.

The resulting cloth or material with the sound absorbing layer is preferably flexible so that it may be configured to function as a boundary to unwanted sound, separating an environment in which there is an undesirable sound level (such as an external environment) from an environment where a lower ambient sound level is desired (or required). Such an internal environment can be created by substantially enclosing the immediate space of an individual with the sheet material soundproofed with the sound absorbing layer.

Of course, the internal environment enclosed with the sheet material covered with the sound absorbing layer also operates to absorb sound generated in the internal environment so that the noise level therein is lowered substantially. This will be useful in public instances such as a user listening to headphones at a loud volume or snoring.

When this same configuration is also intended to enhance the audibility of speech, cup shaped inserts, or passive amplifiers, are secured to the acoustic material. This configuration has an absorber reducing noise entering the garment but amplifying the sound in the direction that the head faces. These cup shaped inserts surround the ear except for an opening directed to what is before the user's eyes/face, similar in concept to horse blinders. Sound directed to the user is readily received at the opening leading directly to the ear.

Another configuration these passive amplifiers are turned to enclose the ear from the front, top and bottom. In this configuration the ear is further protected from sounds entering the hooded sound reduced zone.

### BRIEF DESCRIPTION OF THE DRAWINGS

The invention will be described in conjunction with the following drawings in which like reference numerals designate like elements and wherein:

FIG. 1A Depicts a plan view of a surface of a sound attenuating sheet material 10 formed by adhering sound absorbing material 12 to approximately one half of one surface of the sheet of material 14;

FIG. 1B Shows a side cutaway view of the sheet 10 along the cut A-A′ depicted in FIG. 1A;

FIG. 1C depicts a side cutaway view of a sheet of sound attenuating cloth or material 10′, that includes a sound barrier layer 13 sandwiched between a base layer 14 and a layer of sound-absorbing material 12 (top layer), which top layer is preferably most proximate the user's ear;

FIG. 1D depicts a side cutaway view of a sheet of sound absorbing cloth or material 10″ formed with a sound absorbing layer 12 sandwiched between respective base layers 14;

FIG. 1E depicts a side cutaway view of a material sheet of sound absorbing cloth or material 10′″ formed to include a layer of sound barrier material 13 sandwiched between the layer 12 of sound absorbing material and base layers 14;

FIG. 1F depicts a side cutaway view of a material sheet of sound absorbing cloth or material 10″″ formed to include a sound absorbing layer 12 surrounded on both sides by respective barrier layers 13, upon which respective base layers 14 are provided.

FIG. 1G depicts a side cutaway view of a sound attenuating cloth or material 10′″″, constructed as shown to include a sound barrier layer 13 sandwiched between an upper layer of sound absorbing material 12 and a base layer 14, where the base layer 14 is positioned upon a second sound absorbing layer 12.

FIG. 1H depicts a side cutaway view of a sound attenuating cloth or material 10″″″ constructed as shown to include a sound absorbing layer 12 sandwiched between two barrier layers 13, where the lower of the two barrier layers is positioned on a base layer 14. Preferably, layer 14 is arranged most proximate the user's ear.

FIG. 1I depicts a side cutaway view of a sound attenuating cloth or material 10′″″″ constructed as shown to include a sound barrier layer 13 sandwiched between two sound absorbing layers 12, where the lower of the two sound-absorbing layers is positioned on a base layer 14, which preferably is proximate the user's ear.

FIG. 2 Depicts a hooded sweatshirt (or hoodie) constructed with a sound attenuating cloth prepared to include sound absorbing material on one side (as described above), at least in a head portion;

FIG. 3 Shows a top garment portion of a hooded sweatshirt that includes small cups proximate a wearer's ears to receive and direct sound; and

FIG. 4 Shows a hat with a garment section that is formed with a layer of sound absorbing material that includes small cups proximate a wearer's ears to receive and direct sound.

### DETAILED DESCRIPTION OF THE INVENTION

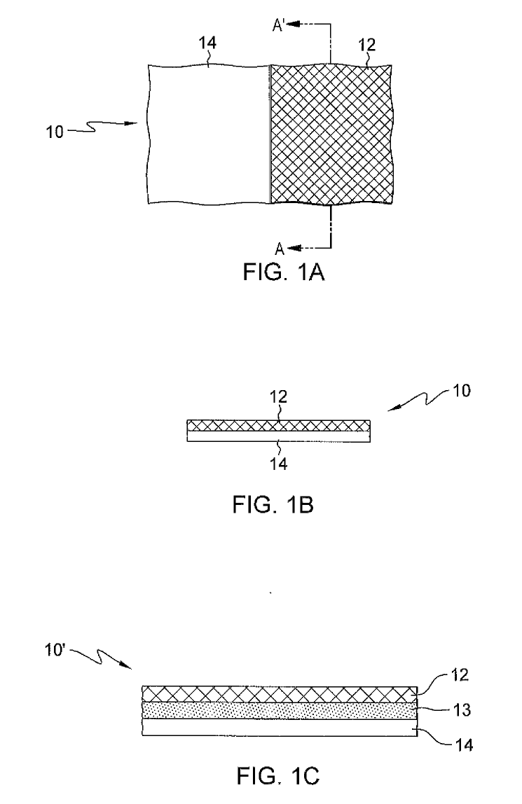

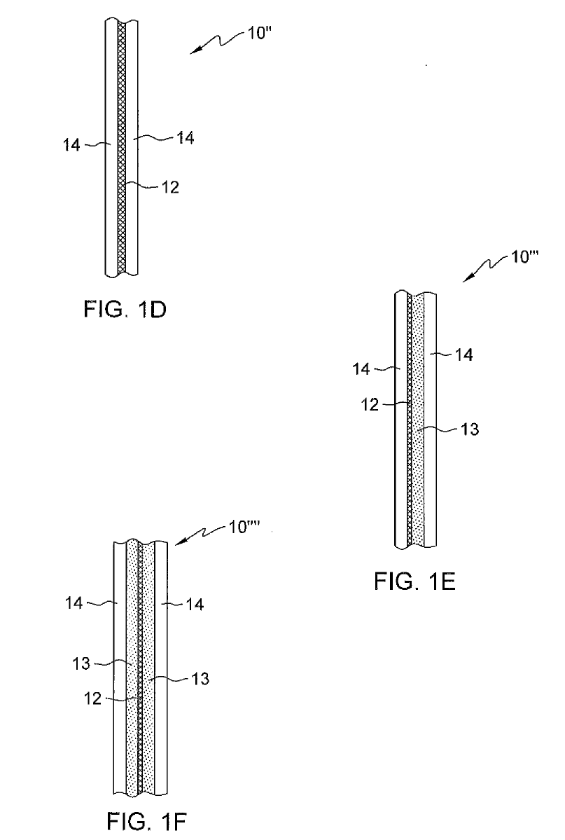

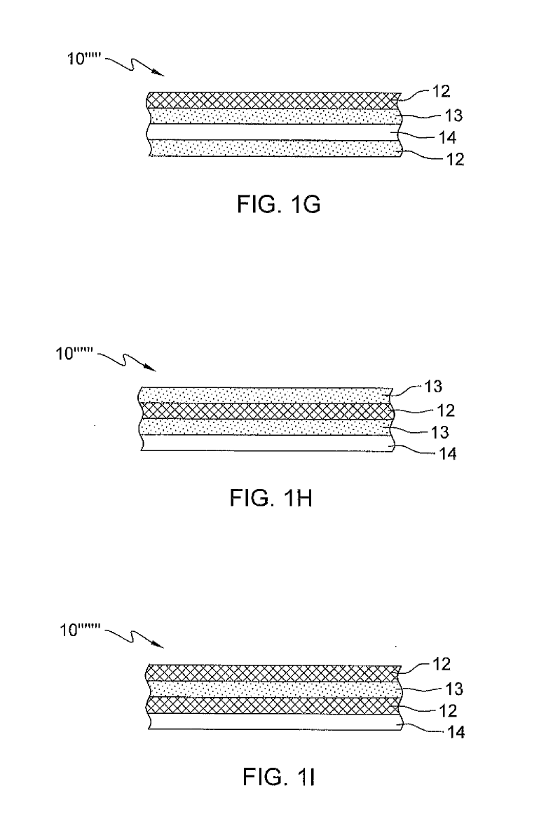

The following is a detailed description and drawings of example embodiments of the invention depicted. The example embodiments are presented in such detail as to clearly communicate the invention and are designed to make such embodiments obvious to a person of ordinary skill in the art. The amount of detail offered is not intended to limit the anticipated variations of embodiments, but to cover all modifications, equivalents, and alternatives falling within the spirit and scope of the present invention, as defined by the appended claims. FIG. 1A depicts a plan view of one embodiment of a sound attenuating cloth or material 10, formed as a sheet. The sheet of the inventive sound-attenuating cloth or material 10 is formed with a layer of sound absorbing material 12 covering a base layer or sheet of material 14. The portion of the base layer 14 is shown only to be partially covered with the layer of sound absorbing material 12, merely to highlight the separate layers. Preferably, the base layer 14 is fully covered by the layer of sound absorbing layer. FIG. 1B shows a side cutaway view of the sound attenuating material 10 along the cut A-A′ depicted in FIG. 1A, showing the layer of sound absorbing material 12 positioned on the base layer 14.

FIG. 1C depicts a side cutaway view of an alternative embodiment of the inventive sound attenuating cloth or material 10′. That is, a sheet of sound attenuating material 10′ is shown in FIG. 1C includes a sound barrier layer 13 sandwiched between a base layer 14 and a layer of sound-absorbing material 12, as shown. Please note that while FIG. 1C shows the sound barrier layer 13 positioned on the base layer 14, with the layer of sound-absorbing material 12 position upon the barrier layer 13, the invention is not limited thereto. For example, the barrier layer 13 may be positioned on the sound absorbing layer 12, which is positioned directly on the base layer 14, or the base layer 14 may be positioned on the sound absorbing layer 12 or the barrier layer 13. Additionally, it is possible that the absorber and barrier are one material and the composition of this material itself acts as the base for a specific application.

FIG. 1D shows an embodiment of the inventive cloth or material sheet formed with a layer of sound absorbing material 12 surrounded by two outer base layers 14. The layers 12, 14 may be said to be sheets, for example. As shown, an inner material (base) layer 14 (formed for its look and feel) and an outer material (base) layer 14 surrounding the sound absorbing layer 12. In a different form, there may be multiple sound absorbing layers 12 surrounded by opposing base layers 14. In that case, the multiple sound absorbing layers 14, together surrounded by the base layers, operate as a whole as a quasi sound barrier layer, such as described below. In that case, the sound absorbing layer 12 is replaced by 1 to 6 or more layers of Lumitex material or other absorbers such as, wool, foam or fiberglass, to form the quasi sound barrier layer. The outer material layer 14 defines the look and feel of the material sheets, for example, by its softness or ability to be written on.

FIG. 1E depicts a side cutaway view of a material sheet of sound absorbing cloth or material 10′″ formed to include a layer of sound barrier material 13 sandwiched between the layer 12 of sound absorbing material and a base layer 14. A second base layer 14 is provided on the layer of sound absorbing material 12, as shown. Accordingly, the cloth or material comprises an inner sound absorbing layer 12 and sound barrier layer 13 surrounded by the base layers. FIG. 1F depicts sound absorbing cloth or material 10″″ formed to include a sound absorbing layer 12 surrounded on both sides by respective barrier layers 13, upon which respective base layers 14 are provided.

Alternatively, the invention provides a sound attenuating cloth or material 10′″″, constructed as shown in FIG. 1G, to include a sound barrier layer 13 sandwiched between a sound absorbing layer 12 positioned upon a base layer 14 and an upper layer of sound absorbing material 12, where the base layer 14 is positioned upon a second sound absorbing layer 12. Of course the reverse order is possible. FIG. 1H depicts a sound attenuating cloth or material 10″″″ comprising a sound absorbing layer 12 sandwiched between two barrier layers 13, where the lower of the two barrier layers is positioned on a base layer 14. Of course the reverse order is possible.

FIG. 1I shows a sound attenuating cloth or material 10′″″″, comprising a sound barrier layer 13 sandwiched between two sound absorbing layers 12, where the lower of the two sound-absorbing layers is positioned on a base layer 14. Of course the reverse order is possible. For that matter, the inventive sound attenuating material or cloth may comprise multiple sound absorbing layers in any laminate arrangement with any number of barrier and/or base layers, without deviating from the scope and spirit of the invention.

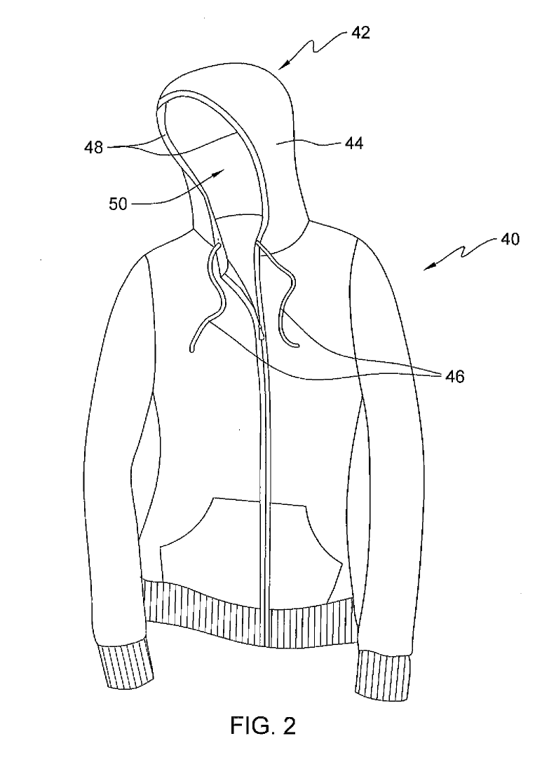

FIG. 2 depicts an exemplary garment in the form of a hooded sweatshirt (or hoodie) 40 constructed with a sound attenuating cloth prepared to include a layer of the sound absorbing material on one side of a base layer (as described above), at least in a head portion 42 of the sweatshirt 40. The sound absorbing material is on the inner surface of sound-attenuating cloth 44, from which the head portion is made. Alternatively, the sound attenuating cloth may be constructed as shown in any of the figures to include a sound barrier layer 13 sandwiched between a base layer 14 and a layer of sound absorbing material 12 (in FIG. 1 C, for example, as shown), etc. The head portion 44 may be integral with the remainder of the hooded sweatshirt, or may be detachable using a zipper, Velcro, etc., without limitation. For that matter, the sound-attenuating cloth or materials (10, 10′, 10″, 10′″, 10″″, 10′″″, 10″″″ and 10′″″″) may comprise a hood portion 42, may be attached directly into the hood portion made of common material such as cotton, or may be a removable (separable, for example, using Velcro), both the lining and the hood itself.

During use, the user can optimize the efficacy of the hood by tightening strings 46, which are part of the head portion 42 formed with the sound attenuating cloth or material 10 with the sound absorbing layer 12 on one side (the inner side of the head portion as shown), material 10′ a base layer 14 on an outer side of the head portion (FIG. 1A, 1B) with or without a barrier layer 13 therebetween (FIG. 1C), or any of the other inventive embodiments of the sound attenuating cloth or material (see, for example, FIGS. 1D, 1E, 1F, 1G and 1F, without limitation), pressing the edges of the head portion 48 against the user's face 50. These pull strings can be of any diameter or width and can also have a further closure attachment at the neck. By substantially closing the space using the strings, the sound-reduced inner volume is formed. Such movement forms a quasi-barrier to some sounds (in reliance on the sound absorbing quality defined by the composition of the sound attenuation cloth or material comprising the head portion) that might otherwise enter the space surrounded by the head portion 48 and proximate the ears and/or move through the sound attenuating material covering the ears.

The hooded sweatshirt 40 (with or without a zipper), as would any other garment constructed with the sound-attenuating cloth (as shown and described herein), has a look and feel like a normal sweatshirt, but the hidden design feature serves to reduce noise levels typically to the person wearing the hooded sweatshirt when in loud settings, such as public transportation, communal study environments, etc., without limitation, but the hooded sweatshirt can also serve the purpose of reducing the sounds made by the person wearing the hoodie to those that near the person.

An acoustic hood, hat or scarf designed with the inventive sound attenuating cloth (s) to noticeably reduce surrounding noise has unlimited possibilities. Some examples are:
a. Hooded garments, such as a sweatshirt, could be worn on planes buses, public transportation or any public space to facilitate the wearer sleeping, reduce disturbance from a crying child or conversation occurring nearby, promote concentration while reading, or to enhance headphones audibility, etc.
b. Open work/office spaces where one is unable to control the volume of environmental noise that is considered to be a distraction and would inhibit productivity.
c. Educational environments where certain noises may be distracting and cause the reduction in retention of read material, or ability to focus on an exam, etc.
d. Autistic children or adults with noise sensitivity who rely on sensory control in unpredictable public environments, such as street, shopping malls, etc.
e. People of ill health, such as those with thyroid problems where noise sensitivity disturbs their sense of well-being.

Individuals that are regularly exposed to high noise levels due to their occupation, such as those employed in the music industry, aircraft industry or in a factory, would clearly benefit from a reduction in noise to which they might otherwise be exposed to noise levels that cause hearing loss in the workplace. Hereto before, such known high-noise vocations provided little to no opportunity for employees to avoid noise found there at substantially higher volumes or frequencies than those recommended by OSHA. Often, hearing protection such as earplugs is required in high noise environments. But such known hearing protection devices are not always used. Earplugs, for example, are easy to misplace or are found to be uncomfortable to users, particularly if used over the long term. NOTE: all garments in the noise reduction only category come with the criteria range from reducing the noise to inhibiting the noise. Additionally, there is the application where someone may be snoring on a plane or talking on their cell phone and these sounds bother other people and the hoodie in the passive muffler state will organically help to reduce those sounds that might disturb people in proximity also.

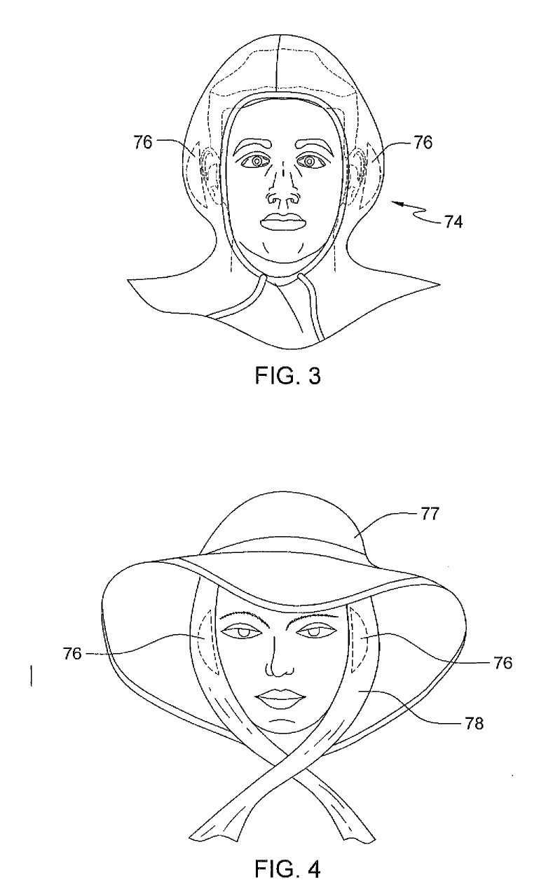

An acoustic hood or hat with small cups to improve hearing clarity is an excellent support for the hearing impaired even without the assistance of a hearing aid. Alternatively, with a hearing aid the hooded sweatshirt can help to focus the sound to the ears, while inhibiting background noise from the sides or back of the person. FIG. 3 shows a top garment portion 74 of a hooded sweatshirt that includes small cups 76 proximate a wearer's ears to receive and direct sound into or away from the ears. This version can be used with, or independent of, an acoustic hood for noise reduction with small speakers in the ear area, eliminating the need for earbuds or headphones. These speakers can be connected with a hardwire, or wirelessly. These speakers also can be incorporated into a hearing aid system.

An acoustic hood for noise reduction or hearing amplification can also provide radio frequency (RF) protection. In an embodiment, in combination with a layer of sound absorbing material provided on a base layer of a sheet material, and a layer or film of a material that attenuates RF energy, at least in some frequency ranges. Such garments may be helpful in minimizing exposure to RF and similar frequency wave energy, for example, in a region with multiple overhead power lines or radio towers.

Please note that while the embodiment shown in FIGS. 1A, 1B was formed with a cloth 10 with only a single noise or sound absorbing layer 12, the sheet of cloth or material 10 may also include multiple noise or sound absorbing layers on one or the other opposing surfaces of the base layer 14 without deviating from the scope and spirit of the invention. The second or additional noise absorbing layer can function as a decoupler for potential low frequency noises or increase the NRC and/or the STC of the sound absorbing layer. The sound waves emitted from any source proximate the wearer (or a portion thereof) are absorbed by the noise absorbing layer before it enters any reduced sound compartment, like a small space for speaking on a cell phone enclosed within the acoustically treated portion of the hood (enclosure). With such a construction, a suitable sound transmission loss is achieved.

The outer, inner or both noise absorbing layers preferably display a high NRC rated material, where “NRC” stands for noise reduction coefficient and represents the average amount of sound absorbed by the material. The NRC rating typically ranges from 0.01 to 1.0. NRC ratings above 1 (e.g., 1.03) are also possible. The higher the NRC rating of the material, the greater the sound absorption of the material. The noise or sound absorbing layer(s) is/are chosen based upon the characteristics of the particular mechanism and noise reduction goals.

Various materials are contemplated. In particular, the material forming the noise or sound absorbing layer(s) can be chosen for sound absorbing qualities and ability to operate as a heat insulator to affect its properties as a garment, maintain heat. As such, the material forming the layer for noise attenuation can differ from that forming the base layer. The sound absorbing layers also may be formed from a closely woven textile-like material, embodying any suitable material provided the material has adequate sound absorbing properties and withstands a predetermined temperature. A suitable adhesive can be used to secure the sound or noise absorbing layer to a base material layer. When multiple noise absorbing layers are provided, the layers can be formed by either the same material or a different material. Alternatively, the noise absorbing layer can be connected to a base or a barrier layer (or multiple barrier layers) when the garment is formed. The connection between layers can be achieved specified to the design or function of the garment, and is not limited to methods of bonding, layering or quilting.

FIG. 4 shows a hat 77 with a garment section 78 that is formed with a layer of sound absorbing material. As in the FIG. 3 embodiment, small cups 76 are included within the garment section 78.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. A garment engaged in a sound-reducing state, comprising:
a first garment portion, the first garment portion formed from a sound attenuating cloth that comprises a base layer, a layer of sound barrier material provided on the base layer and a layer of sound-absorbing material is provided on the sound barrier layer and configured to surround part of a wearer's head and form a substantially enclosed reduced-sound zone about the user's ears when the first garment portion is engaged in a sound-reducing state; and
a second garment portion operable with the first garment portion;
wherein in the sound-reducing state, the layer sound-absorbing material and the sound barrier layer together substantially reduce an amount of sound passing over and through the sound attenuating cloth into the substantially enclosed reduced-sound zone.

2. The garment of claim 1, further comprising a second sound-absorbing layer provided between the base layer and the sound barrier layer.

3. The garment of claim 1, further comprising a second base layer provided on the sound absorbing layer.

4. The garment of claim 1, wherein the base layer is a natural or synthetic material.

5. The garment of claim 1, wherein the first garment portion is detachably connected to the second garment portion.

6. The garment of claim 5, comprising a hooded sweatshirt.

7. The garment of claim 6, wherein the first garment portion is a detachable hood and wherein the detachable hood is deployed to form the substantially enclosed reduced-sound zone.

8. The garment of claim 1, wherein the first garment portion and the second garment portion are detachably connected using a zipper or Velcro.

9. The garment of claim 1, wherein the reduced-sound zone minimizes an amount of sound entering the reduced-sound zone from outside the garment and an amount of sound exiting the zone from inside the garment.

10. The garment of claim 1, further including small cups positioned in or proximate the reduced-sound zone in an arrangement for receiving and directing sound towards the wearer's ears.

11. The garment of claim 10, wherein the small cups are arranged to be tight to the wearer's ear at any of the back, front, top and bottom of the small cups.

12. The garment of claim 11, wherein the small cups are open and maintain a space between a face of the wearer and a wall of the first garment portion, the space for receiving and/or amplifying sound directed to the wearer.

13. The garment of claim 1, further including small cups in or proximate the reduced-sound zone, for inhibiting sound going towards the wearer's ears.

14. The garment of claim 13, wherein the small cups are arranged to be tight to the wearer's ear all around the small cups.

15. The garment of claim 1, wherein the first garment portion is a scarf and wherein the scarf is engaged to form the substantially enclosed reduce-sound zone.

16. The garment of claim 15, wherein the scarf is part of another system, such as a hat or cape.

17. The garment of claim 1, wherein the first garment portion is a hood and wherein the hood is configured to be substantially forward of a face of the wearer's head, creating a passive muffler.

18. The garment of claim 1, wherein the first garment portion is a hood and wherein the hood is configured to be closed, creating a passive muffler.

19. A hooded sweatshirt or cape engaged in a sound-reducing state, comprising:
a first garment portion formed as a hood portion of the sweatshirt or cape from a sound attenuating cloth that comprises a base layer upon which a sound barrier layer is provided and at least one layer of sound absorbing material provided on either the base layer or on the sound barrier layer, wherein the first garment portion is configured to surround part of a wearer's head and form a substantially enclosed reduced-sound zone about the user's ears when the first garment portion is engaged in a sound-reducing state; and
a second garment portion formed as a trunk portion of the sweatshirt or cape that is operable with the first garment portion;
wherein in the sound-reducing state, the Lumitex® sound barrier/absorber layer and the at least one layer of sound absorbing material together substantially block and prevent sound from passing through and over the sound attenuating cloth into the substantially enclosed reduced-sound zone.

20. A hooded sweatshirt or scarf arranged to effect a sound-reducing state, comprising:
a first garment portion formed as a hood portion of the sweatshirt or scarf from a sound attenuating cloth that comprises a base layer upon which a sound barrier layer is provided, wherein at least one layer of sound absorbing material is provided on either the base layer or on the sound barrier layer and wherein the first garment portion is configured to surround part of a wearer's head and form a substantially enclosed reduced-sound zone about the user's ears when the first garment portion is engaged in a sound-reducing state; and
a second garment portion formed as a trunk portion of the sweatshirt or scarf that is operable with the first garment portion;
wherein in the sound-reducing state, the sound barrier layer and the sound absorbing material together substantially block and prevent sound from passing through and over the sound attenuating cloth into the substantially enclosed reduced-sound zone.
